(19) United States
(12) Patent Application Publication
(10) Pub. No.: US 2006/0148810 A1
(43) Pub. Date: Jul. 6, 2006

(54) Treatment of amm

(76) Inventor: Frank Giles, Bellaire, TX (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/530,451

(22) PCT Filed: Oct. 9, 2003
(86) PCT No.: PCT/EP03/11214

§ 371 Date: Oct. 4, 2005

Related U.S. Application Data
(60) Provisional application No. 60/417,622, filed on Oct. 10, 2002.

(51) Int. Cl. A61K 31/501

(52) U.S. Cl. 514/252.03

(57) ABSTRACT

The present invention relates to a method of treating a warm-blooded animal, especially a human, having agnogenic myeloid metaplasia (AMM), especially agnogenic myeloid metaplasia which is resistant to conventional chemotherapy.

The present invention relates to a method of treating a warm-blooded animal, especially a human, having agnogenic myeloid metaplasia (AMM), especially agnogenic myeloid metaplasia which is resistant to conventional chemotherapy, comprising administering to said animal a therapeutically effective amount of a 4-pyridylmethyl-phthalazine derivative, especially a compound of formula I as defined herein, alone or in combination with further therapeutic agents, for example, those defined herein; the use of a 4-pyridylmethyl-phthalazine derivative alone or in combination with further therapeutic agents, for example, those defined herein, for the preparation of a medicament for the treatment of agnogenic myeloid metaplasia; to a combination comprising a 4-pyridylmethyl-phthalazine derivative, a further therapeutic agent as defined herein and optionally at least one pharmaceutically acceptable carrier, for simultaneous, separate or sequential use; and to a pharmaceutical composition and a commercial package comprising said combination.

Agnogenic myeloid metaplasia (AMM, also known as idiopathic myelofibrosis) is one of a group of blood cell diseases that originates from mutation or change in the DNA of a single stem cell in the bone marrow. The DNA damage in a single stem cell that is normally capable of forming all different blood cells has the effect that too few red cells and usually too many white cells and platelets are produced. The average survival time after diagnosis of AMM is about five years. About 10% of patients having AMM are at risk of developing acute myelogenous leukemia (AML). AML is a bone marrow malignancy where the marrow is replaced by a population of extremely immature or primitive “blast” or stem cells.

Patients having AMM usually experience vague and non-specific symptoms like weakness, fatigue, shortness of breath and a fullness or a dragging sensation in the left upper part of the abdomen. An enlarged spleen is virtually a constant finding and sometimes said enlargement is dramatic. Marrow fibrosis is seen in about all patients with AMM.

For further diagnosis of AMM, a complete blood cell count is done. If it is confirmed that a patient has a decrease in red blood cells (anemia) and an increase in white blood cells and platelets, a bone marrow examination is performed.

To some AMM patients low doses of chemotherapy are given, especially low doses of hydroxyurea, in order to decrease very high platelet counts or the size of the spleen. However, there is no assurance that chemotherapy with established chemotherapeutic agents or other forms of therapy will be beneficial to the patient. Therefore, there is a strong need for further chemotherapeutic agents for the treatment of AMM, especially for the treatment of AMM that is resistant to chemotherapy with established chemotherapeutic agents.

Surprisingly, it was found that 4-pyridylmethyl-phthalazine derivatives are useful for the treatment of AMM.

4-Pyridylmethyl-phthalazine derivatives which a suitable for the present invention, their preparation and pharmaceutical formulations containing the same are described in WO00/59509, EP02/04892, WO01/10859 and, especially, in U.S. Pat. No. 6,258,812, which are here incorporated by reference.

Compounds that are also preferred for the treatment of AML according to the present invention are those generically or specifically disclosed, mentioned or generically and specifically claimed in EP 1 259 487, WO 01/55114, EP 1 129 075, WO 00127820, EP 1 107 964, WO 00/09495, EP 1 165 085, WO 02/090343, WO 01/85715, WO 01/85691, WO 02/092603, WO 03/040101 and WO 03/040102, the entire contents of which hereby are incorporated by reference.

In particular compounds which target, decrease or inhibit the activity or production of VEGF are those compounds, proteins, aptamers or monoclonal antibodies generically and specifically disclosed in WO 98/45331, WO 98/11223, WO 00/27819 and EP 0 769 947; those as described by M. Prewett et al in Cancer Research 59 (1999) 5209-5218, by F. Yuan et al in Proc. Natl. Acad. Sci. USA, vol. 93, pp.14765-14770, Dec.1996, by Z. Zhu et al in Cancer Res. 58, 1998, 3209-3214, and by J. Mordenti et al in Toxicologic Pathology, Vol. 27, no. 1, pp 14-21, 1999; in WO 00/37502 and WO 94/10202; Angiostatin™, described by M. S. O'Reilly et al, Cell 79, 1994, 315-328; Endostatin™, described by M. S. O'Reilly et al, Cell 88, 1997, 277-285; anthranilic acid amides; ZD4190; ZD6474; SU5416; SU6668; anti-VEGF antibodies or anti-VEGF receptor antibodies, e.g. RhuMab; or those as described in U.S. Pat. Nos. 6,168,778, 6,147,204, 6,051,698, 6,011,020, 5,958,691, 5,817,785, 5,811,533, 5,696,249, 5,683,867, 5,670,637, and 5,475,096, e.g. pegaptanib sodium, are also preferred for the treatment of AMM according to the present invention.

4-Pyridylmethyl-phthalazine derivatives and, in particular 4-pyridylmethyl-phthalazine derivatives of formula I, 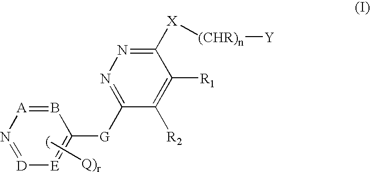
wherein the radicals and symbols have the meanings as defined below, the N-oxides of these 4-pyridylmethyl-phthalazine derivatives, as well as the salts thereof, are tyrosine kinase inhibitors, which were designed to inhibit the vascular endothelial growth factor (VEGF) signal transduction by binding directly to the ATP-binding sites of VEGF receptors. Such 4-pyridylmethyl-phthalazine derivatives reduce the microvasculature and inhibit growth of primary tumors and metastases in animal models and are useful for treating diseases associated with deregulated angiogenesis, especially neoplastic diseases (solid tumors), such as breast cancer, cancer of the colon, lung cancer, especially small cell lung cancer, and cancer of the prostate.

For example, PTK787 (also known as ZK222584), a compound of formula I, wherein r, n and m are each 0, R₁ and R₂ together form a bridge of subformula I*, A, B, D and E are each CH, G is methylene, X is imino, Y is 4-chlorophenyl, and the bonds characterized by a wavy line are double bonds, is most specific for KDR, but can also inhibit Flt-1 and Flt-4 and has activity against other tyrosine kinase receptors, including c-Kit, bFGF and PDGF.

Hence, the invention relates to a method of treating AMM, especially AMM which is resistant to conventional chemotherapy, comprising administering a therapeutically effective amount of a 4-pyridylmethyl-phthalazine derivative to a warm-blooded animal in need thereof, preferably of a therapeutically effective amount of a 4-pyridylmethyl-phthalazine derivative of formula I, wherein

r is 0 to 2,

n is 0 to 2,

m is 0 to 4,

R₁ and R₂ (i) are lower alkyl or

(ii) together form a bridge in subformula I* 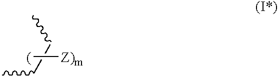
the binding being achieved via the two terminal carbon atoms, or

(iii) together form a bridge in subformula I** 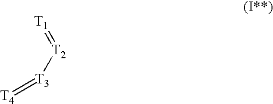
wherein one or two of the ring members T₁, T₂, T₃ and T₄ are nitrogen, and the others are in each case CH, and the binding is achieved via T₁ and T₄;

A, B, D, and E are, independently of one another, N or CH, with the stipulation that not more than 2 of these radicals are N;

G is lower alkylene, lower alkylene substituted by acyloxy or hydroxy, —CH₂—O—, —CH₂—S—, —CH₂—NH—, oxa (—O—), thia (—S—), or imino (—NH—);

Q is lower alkyl;

R is H or lower alkyl;

X is imino, oxa, or thia;

Y is unsubstituted or substituted aryl, pyridyl, or unsubstituted or substituted cycloalkyl; and

Z is amino, mono- or disubstituted amino, halogen, alkyl, substituted alkyl, hydroxy, etherifled or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;

and wherein the bonds characterized, if present, by a wavy line are either single or double bonds;

or an N-oxide of the defined compound,

or the salt of such compound having at least one salt-forming group.

The radicals and symbols as used in the definition of a compound of formula I have the meanings as disclosed in WO 98/35958 which publication is hereby incorporated into the present application by reference.

A preferred compound of formula I is PTK787. More preferably, PTK787 is employed in the form of its succinate salt.

It will be understood that in the discussion of methods, references to the active ingredients are meant to also include the pharmaceutically acceptable salts. If these active ingredients have, for example, at least one basic center, they can form acid addition salts. Corresponding acid addition salts can also be formed having, if desired, an additionally present basic center. The active ingredients having an acid group (for example COOH) can also form salts with bases. The active ingredient or a pharmaceutically acceptable salt thereof may also be used in form of a hydrate or include other solvents used for crystallization.

The term “treatment” as used herein comprises the treatment of patients having AMM or being in a pre-stage of said disease which effects the delay of progression of the disease in said patients.

A complete response to the treatment can, e.g., be defined by a white blood cell count between 1 to 10×10⁹/L with no peripheral blasts, promyelocytes, or myelocytes and with normalization of bone marrow differential (<5% blasts in normocellular or hypercellular marrow).

A partial response to the treatment can, e.g., be defined by a white blood cell count between 1-10×10⁹/L with persistence of immature cells (blasts, myelocytes, metamyelocytes) for pretreatment leukocytosis.

For the treatment of AMM a 4-pyridylmethyl-phthalazine derivative can be administered alone or in combination with other forms of treatments, e.g. splenectomy, stem cell transplantation in case a well-matched donor is available, or administration of other therapeutic agents including the following agents. Androgens, like oxymethalone or danazol, or prednisone can be administered to patents not producing enough red blood cells. Hydroxyurea can be used in order to help decreasing very high platelet and white blood cell counts or the size of the spleen.

Hence, the present invention pertains also to a combination comprising a 4-pyridylmethylphthalazine derivative, preferably a compound of formula I as defined above, and at least one compound selected from the group consisting of an androgen, prednisone and hydroxyurea, in which the active ingredients are present in each case in free form or in the form of a pharmaceutically acceptable salt and optionally at least one pharmaceutically acceptable carrier, for simultaneous, separate or sequential use, especially for use in a method of treating AMM

A combination comprising a 4-pyridylmethyl-phthalazine derivative and a compound and at least one compound selected from the group consisting of an androgen, prednisone and hydroxyurea, in which the active ingredients are present in each case in free form or in the form of a pharmaceutically acceptable salt and optionally at least one pharmaceutically acceptable carrier, will be referred to hereinafter as a COMBINATION OF THE INVENTION.

The COMBINATION OF THE INVENTION can be a combined preparation or a pharmaceutical composition.

The term “a combined preparation”, as used herein defines especially a “kit of parts” in the sense that the active ingredients as defined above can be dosed independently or by use of different fixed combinations with distinguished amounts of the ingredients, i.e., simultaneously or at different time points. The parts of the kit can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the active ingredients. The ratio of the total amounts of the active ingredient 1 to the active ingredient 2 to be administered in the combined preparation can be varied, e.g., in order to cope with the needs of a patient sub-population to be treated or the needs of the single patient which different needs can be due to age, sex, body weight, etc. of the patients. Preferably, there is at least one beneficial effect, e.g., a mutual enhancing of the effect of the first and second active ingredient, in particular a synergism, e.g. a more than additive effect, additional advantageous effects, less side effects, a combined therapeutical effect in a non-effective dosage of one or both of the first and second active ingredient, and especially a strong synergism the first and second active ingredient.

Additionally, the present invention provides a method of treating AMM comprising administering a COMBINATION OF THE INVENTION in an amount which is jointly therapeutically effective against AMM to a warm-blooded animal in need thereof.

The person skilled in the pertinent art is fully enabled to select relevant test models to prove the hereinbefore and hereinafter mentioned beneficial effects on AMM of a 4-pyridylmethylphthalazine derivative or of a COMBINATION OF THE INVENTION. The pharmacological activity of a 4-pyridylmethyl-phthalazine derivative or a COMBINATION OF THE INVENTION may, for example, be demonstrated in a suitable clinical study. Suitable clinical studies are, for example, open label non-randomized, dose escalation studies in patients with advanced AMM. Such studies prove in particular the synergism observed with the COMBINATIONS OF THE INVENTION. The beneficial effects on AMM can be determined directly through the results of such studies or by changes in the study design which are known as such to a person skilled in the art. For example, one combination partner can be administered with a fixed dose and the dose of a second combination partner is escalated until the Maximum Tolerated Dosage (MTD) is reached. Alternatively, a placebo-controlled, double blind study can be conducted in order to prove the benefits of the COMBINATION OF THE INVENTION mentioned herein.

It is one objective of this invention to provide a pharmaceutical composition comprising a quantity, which is jointly therapeutically effective against AMM comprising the COMBINATION OF THE INVENTION. In this composition, the combination partners can be administered together, one after the other or separately in one combined unit dosage form or in two separate unit dosage forms. The unit dosage form may also be a fixed combination.

The pharmaceutical compositions for separate administration of the combination partners and for the administration in a fixed combination, i.e. a single galenical composition comprising at least two combination partners, according to the invention can be prepared in a manner known per se and are those suitable for enteral, such as oral or rectal, and parenteral administration to mammals (warm-blooded animals), including man, comprising a therapeutically effective amount of at least one pharmacologically active combination partner alone or in combination with one or more pharmaceutically acceptable carries, especially suitable for enteral or parenteral application.

Novel pharmaceutical composition contain, for example, from about 10 % to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partner of the COMBINATION OF THE INVENTION may be administered simultaneously or sequentially and in any order, and the components may be administered separately or as a fixed combination. For example, the method of treatment of AMM according to the present invention may comprise (i) administration of a combination partner (a) in free or pharmaceutically acceptable salt form and (ii) adminstration of a combination partner (b) in free or pharmaceutically acceptable salt form, simultaneously or sequentially in any order, in jointly therapeutically effective amounts, preferably in synergistically effective amounts, e.g. in daily dosages corresponding to the amounts described herein. The individual combination partners of the COMBINATION OF THE INVENTION can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

The effective dosage of a 4-pyridylmethyl-phthalazine derivative and of the combination partners employed in the COMBINATION OF THE INVENTION may vary depending on the particular compound or pharmaceutical composition employed, the mode of administration, the type of the AMM being treated, the severity of the AMM being treated and the co-medication. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of a 4-pyridylmethyl-phthalazine derivative or of the single active ingredients of the COMBINATION OF THE INVENTION required to prevent, counter or arrest the progress of the condition. Optimal precision in achieving concentration of the active ingredients within the range that yields efficacy without toxicity requires a regimen based on the kinetics of the active ingredients' availability to target sites.

If the warm-blooded animal is an adult human, the dosage of a compound of formula I, especially PTK787, is preferably in the range of about 250 to 2000, more preferably about 500 to 1750, and most preferably 1000 to 1500, mg/day. Preferably, the total daily amount of the drug is applied in two or more units of equal or different weight. For example, 500 mg or 750 mg of PTK787 can be administered twice daily, or two different units, one having 500 mg of PTK787 and the other 750 mg of PTK787 can be applied on the same day.

Moreover, the present invention provides a commercial package comprising as active ingredients the COMBINATION OF THE INVENTION, together with instructions for simultaneous, separate or sequential use thereof in the treatment of AMM.

The present invention also provides the use of a 4-pyridylmethyl-phthalazine derivative and the use of a COMBINATION OF THE INVENTION for the preparation of a medicament for the treatment of AMM and, still further, for the use of a pharmaceutical composition comprising a 4-pyridylmethyl-phthalazine derivative or a COMBINATION OF THE INVENTION for the treatment of AMM.

### EXAMPLE 1

A patient with splenomegaly obtained 750 mg of PTK787 twice daily. During the first 14 days of chemotherapy a decrease of splenomegaly was observed and a drop in white blood cells count from 19,000 to 14,000. The therapy was interrupted in view of dose limiting toxicity. During the interruption of therapy, the spleen began to increase again.

### EXAMPLE 2

A patient having a protuberant spleen that was so massive that it had reached the other side of the patients abdomen, obtained 750 mg of PTK787 twice daily. After five days of therapy, the spleen had less than 10% of its original size.

The Examples demonstrates that PTK787 is suitable for the treatment of AMM.

1. A method of treating agnogenic myeloid metaplasia comprising administering a therapeutically effective amount of a 4-pyridylmethyl-phthalazine derivative to a warm-blooded animal in need thereof.

2. Method according to claim 1 comprising administering a therapeutically effective amount of a 4-pyridylmethyl-phthalazine derivative of formula I 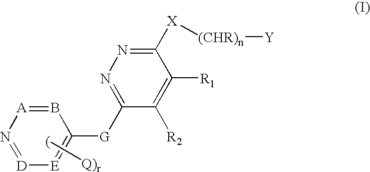
wherein
r is 0 to 2,
n is 0 to 2,
m is 0to4,
R₁ and R₂ (i) are lower alkyl or
(ii) together form a bridge in subformula I* 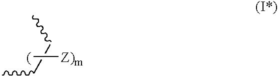
the binding being achieved via the two terminal carbon atoms, or
(iii) together form a bridge in subformula I** 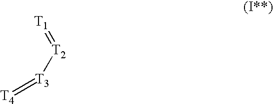
wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound, wherein 1 or more N atoms carry an oxygen atom,
or the salt of such compound having at least one salt-forming group,
to a warm-blooded animal in need thereof.

3. Method of claim 2 wherein the 4-pyridylmethyl-phthalazine derivative of formula I is PTK787.

4. Method according to claim 1 wherein the disease is resistant to conventional chemotherapy.

5. Method according to claim 1 wherein the warm-blooded animal is a human.

6. A combination comprising a 4-pyridylmethyl-phthalazine derivative and at least one compound selected from the group consisting of an androgen, prednisone and hydroxyurea, in which the active ingredients are present in each case in free form or in the form of a pharmaceutically acceptable salt and optionally at least one pharmaceutically acceptable carrier, for simultaneous, separate or sequential use.

7. Combination according to claim 6 wherein the 4-pyridylmethyl-phthalazine derivative is PTK787.

8. Combination according to claim 7 for simultaneous, separate or sequential use in the treatment of agnogenic myeloid metaplasia.

9. A method of treating agnogenic myeloid metaplasia comprising administering a combination as defined in claim 7 in an amount which is jointly therapeutically effective against agnogenic myeloid metaplasia to a warm-blooded animal in need thereof.

10. A pharmaceutical composition comprising a quantity, which is jointly therapeutically effective against agnogenic myeloid metaplasia, of a combination according to claim 7 and at least one pharmaceutically acceptable carrier.

11. A commercial package comprising a 4-pyridylmethyl-phthalazine derivative and at least one compound selected from the group consisting of an androgen, prednisone and hydroxyurea together with instructions for simultaneous, separate or sequential use thereof in the treatment of agnogenic myeloid metaplasia.

12. Use of a 4-pyridylmethyl-phthalazine derivative of formula I 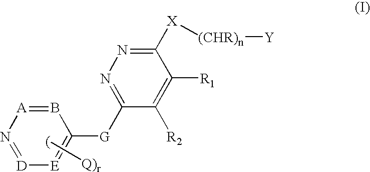
wherein
r is 0 to 2,
n is 0 to 2,
m is 0 to 4,
R₁ and R₂ (i) are lower alkyl or
(ii) together form a bridge in subformula I* 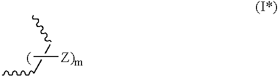
the binding being achieved via the two terminal carbon atoms, or
(iii) together form a bridge in subformula I** 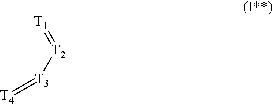
wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound, wherein 1 or more N atoms carry an oxygen atom,
or the salt of such compound having at least one salt-forming group,
optionally in combination with at least one compound selected from the group consisting of of an androgen, prednisone and hydroxyurea, in which the active ingredients are present in each case in free form or in the form of a pharmaceutically acceptable salt and optionally at least one pharmaceutically acceptable carrier for the manufacture of a medicament for the treatment of AMM.

13. Use according to claim 12 wherein the 4-pyridylmethyl-phthalazine derivative of formula I is PTK787.

14. Use of a pharmaceutical composition comprising a 4-pyridylmethyl-phthalazine derivative or a combination according to claim 6 for the treatment of AMM.
